(19) United States
(12) Patent Application Publication
(10) Pub. No.: US 2026/0000371 A1
(43) Pub. Date: Jan. 1, 2026

(54) SIDE-LYING BED FOR X-RAY IMAGING

(71) Applicant: SEOUL NATIONAL UNIVERSITY HOSPITAL, Seoul (KR)

(72) Inventor: Kyung Deok BAE, Suwon-si, Gyeonggi-do (KR)

(21) Appl. No.: 18/992,923

(22) PCT Filed: Jul. 11, 2023
(86) PCT No.: PCT/KR2023/009828

§ 371 Date: Jan. 9, 2025

(30) Foreign Application Priority Data
Jul. 11, 2022 (KR) 10-2022-0085286

(51) Int. Cl. A61B 6/04; A61B 6/00; A61B 6/42; A61G 7/05

(52) U.S. Cl. CPC A61B 6/0407; A61B 6/42; A61B 6/4405; A61B 6/4452; A61G 7/0507; A61G 2200/322; A61G 2210/50

(57) ABSTRACT

The present invention relates to a bed configured to such that a patient with the chest full of water or air may assume a posture of lying on his or her side when taking an X-ray image and, in particular, configured to allow X-ray imaging to be easily carried out when the patient is lying on his or her side, reduce the transportation of a patient with limited mobility, and reduce the staffing of a medical team. The side-lying bed, according to the present invention, to achieve the purposes as described above comprises: a frame that supports a patient with a side-lying posture; a mattress with cushions positioned on the upper surface of the frame; and a detector mounting unit formed on one side of the frame in a longitudinal direction of the frame such that a detector for X-ray imaging can be mounted on the detector mounting unit.

### TECHNICAL FIELD

The present invention relates to a bed configured to allow a patient whose chest is filled with water or air to take a side-lying position during x-ray imaging, and in particular, the bed is configured to allow X-ray imaging to be easily performed in the side-lying position of the patient, reduce the transportation of patients with mobility difficulties, and reduce the manpower of medical staff.

### BACKGROUND ART

In the case of a patient whose chest is filled with water or air, it is generally more effective to carry out X-ray imaging while maintaining a side-lying position on a imaging table as shown in FIG. 1 than when the patient is standing upright during X-ray imaging.

In the drawings, FIG. 1 is a schematic view showing a conventional imaging table installed in an X-ray imaging room.

As shown in FIG. 1, an imaging table 1 is located in an X-ray imaging room, and a detector stand 6 is installed on a side of the imaging table 1 to fix a detector 4 to the detector stand 6, and then X-ray imaging is performed.

Therefore, in order to perform X-ray imaging, a patient needs to move to an X-ray imaging room, and the X-ray imaging is performed in a state of lying on a side on the imaging table.

In order to take an X-ray image of a patient whose chest is filled with water or air, it is necessary to move the patient from a bed in a hospital room to a transfer bed, transfer the transfer bed to an X-ray imaging room, then move the patient from the transfer bed to a imaging table in the X-ray imaging chamber, and then take a side-lying position. And after the X-ray imaging is completed, the patient moves to the hospital room bed in the reverse order.

There is a problem that the pain felt by the patient is considerable as the patient with mobility difficulties moves by moving the bed several times for X-ray imaging, and there is a disadvantage that the medical staff also needs considerable manpower to transport the patient.

In recent years, a lightweight portable X-ray imaging apparatus has been developed, and X-ray imaging is performed by a medical staff moving with the portable X-ray image imaging apparatus without moving a patient.

### DISCLOSURE OF INVENTION

### Technical Problem

The present invention has been made to solve the problems of the prior art as described above, and an object of the present invention is to provide a side-lying bed for X-ray imaging configured such that X-ray imaging may be easily performed by an X-ray imaging apparatus in a state in which a patient lies on his or her side.

### Technical Solution

In order to achieve the above-described object, a side-lying bed for x-ray imaging according to the present invention includes a frame that supports a patient in a side-lying posture, a mattress with cushions positioned on the upper surface of the frame, and a detector mounting unit formed on one side of the frame in a longitudinal direction of the frame such that a detector for X-ray imaging may be mount on the detector mounting unit.

Further, according to a preferred embodiment of the present invention, the detector mounted on the detector mounting unit and positioned upright, and a lower boundary point of the imaging area of the detector is located below the upper surface of the mattress.

Further, according to a preferred embodiment of the present invention, a handrail formed on the other side of the frame is extended to the upper portion of the frame or folded to the lower portion of the frame, and when the handrail is folded, the handrail is located below the top surface of the mattress.

Further, according to a preferred embodiment of the present invention, the detector mounting unit is an elongated groove formed in a longitudinal direction of the frame.

Further, according to a preferred embodiment of the present invention, the detector mounting unit is a rail fixed in a longitudinal direction of the frame, and the detector is inserted and mounted in a gap between the rail and a side surface of the mattress.

Further, according to a preferred embodiment of the present invention, the detector mounting unit is fixed to the frame with two rails parallel to each other, and the detector is mounted by being inserted into the gap formed between the two rails.

Further, according to a preferred embodiment of the present invention, legs of the frame are equipped with wheels and are movable.

Further, according to a preferred embodiment of the present invention, the mattress is made of a synthetic resin capable of transmitting X-rays.

Further, according to a preferred embodiment of the present invention, the detector mounting units are formed on both sides of the frame respectively, and handrails are mounted on the outer sides of the detector mounting units on both sides respectively, and the handrails are extended to the upper portion of the frame or folded to the lower portion of the frame, and when the handrails are folded, the handrails are positioned below the upper surface of the mattress.

### Advantageous Effects

As described above, in the side-lying bed for X-ray imaging according to the present invention, the detector mounting unit capable of mounting the portable detector is formed on one side of the frame, so that X-ray imaging may be performed in a side-lying posture on the bed on which the patient is lying through the portable X-ray imaging device. Therefore, it is advantageous that the patient does not need to move, the convenience of the patient may be increased, and the manpower of the medical staff may be reduced.

In addition, the side-lying bed for X-ray imaging according to the present invention may be configured as a transfer bed in which wheels are mounted on the legs of the bed. In this case, there is an advantage that it is possible to perform X-ray imaging directly on the transfer bed through a fixed X-ray imaging apparatus mounted in the X-ray imaging room without having to transfer to the imaging table of the X-ray imaging room as the prior art.

### BRIEF DESCRIPTION OF DRAWINGS

FIG. 1 is a schematic view showing a conventional imaging table installed in an X-ray imaging room.

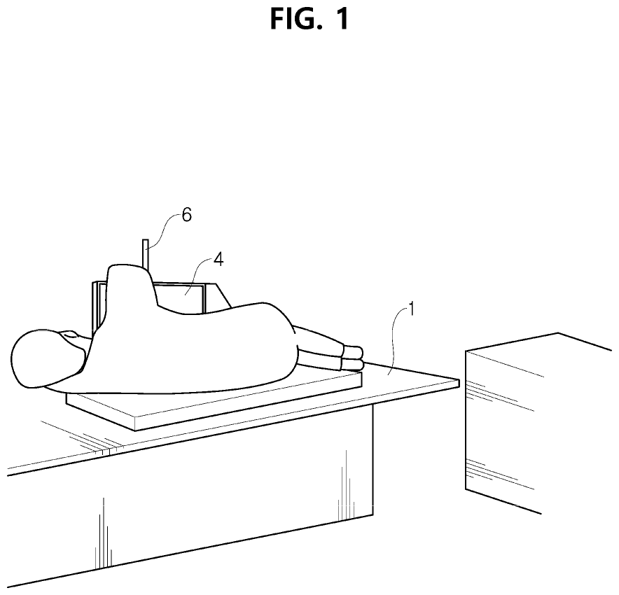

FIG. 2 is a perspective view showing a side-lying bed for X-ray imaging according to the present invention,

FIG. 3 is a cross-sectional view of the bed in a state where a detector is mounted on a detector mounting unit shown in FIG. 2.

FIG. 4 is a schematic view showing the process of taking an X-ray image while a patient is lying on his/her side on the side-lying bed for X-ray imaging shown in FIG. 2.

FIG. 5 is a cross-sectional view of the bed in the state shown in FIG. 4.

FIG. 6 is a schematic view showing another example of the detector mounting unit shown in FIG. 3.

FIG. 7 is a schematic view showing the other example of the detector mounting unit shown in FIG. 3.

FIG. 8 is a schematic view showing an example in which wheels are mounted so that the bed shown in FIG. 2 may be transferred.

### BEST MODE FOR CARRYING OUT THE INVENTION

In the following, a preferred embodiment of a side-lying bed for X-ray imaging according to the present invention will be described in detail with reference to the accompanying drawings.

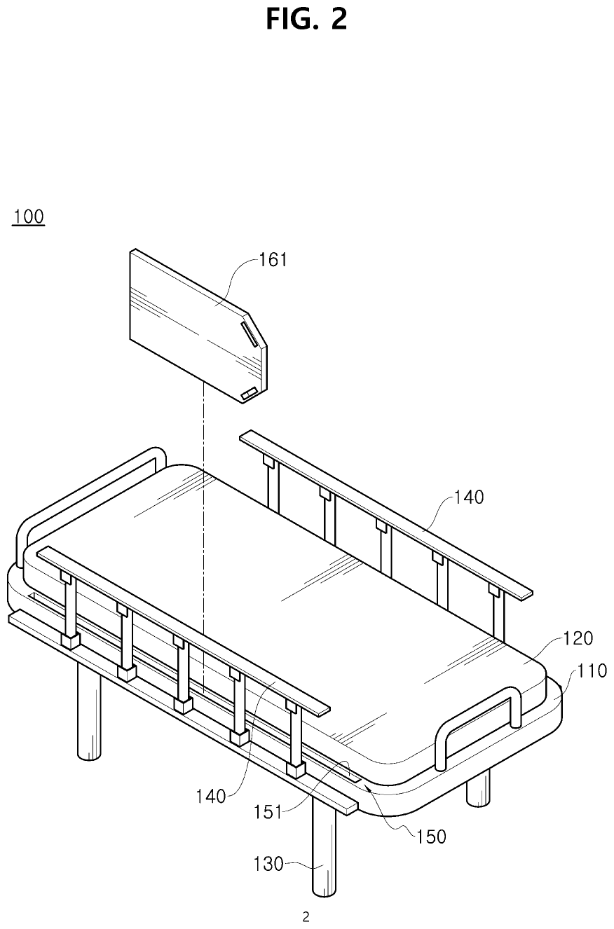

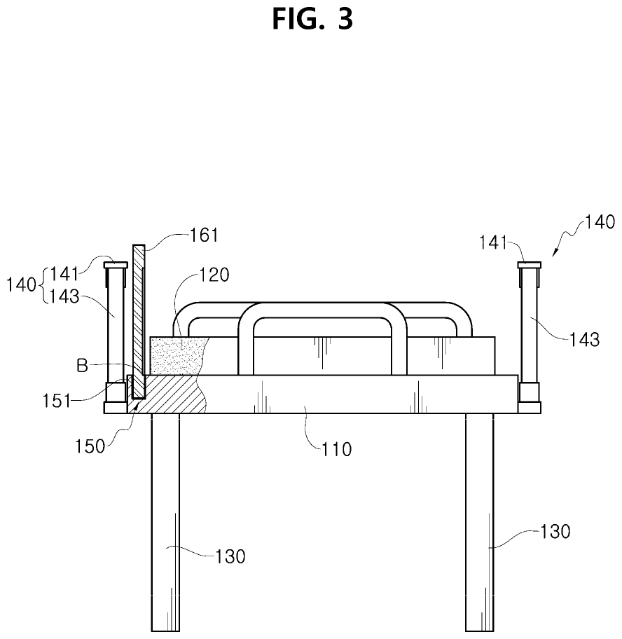

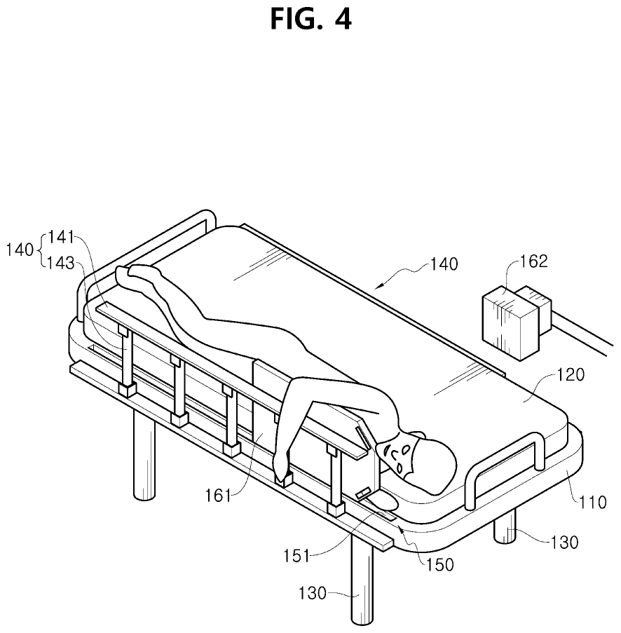

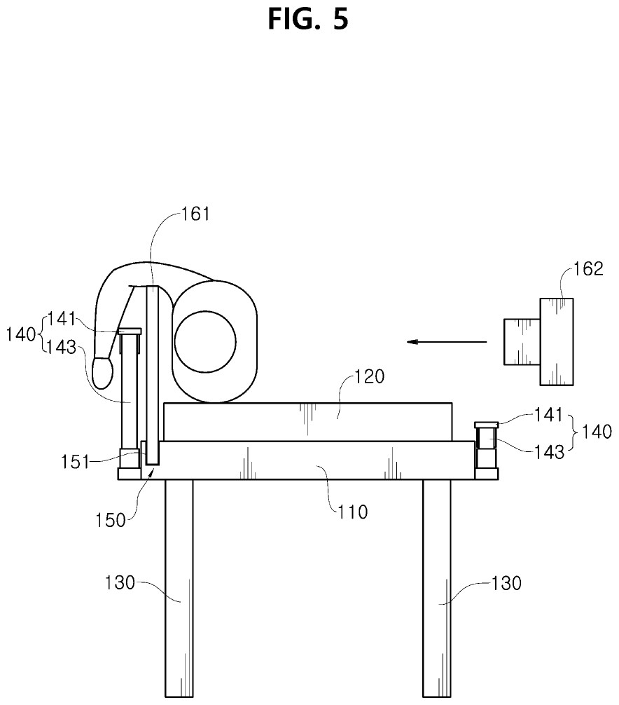

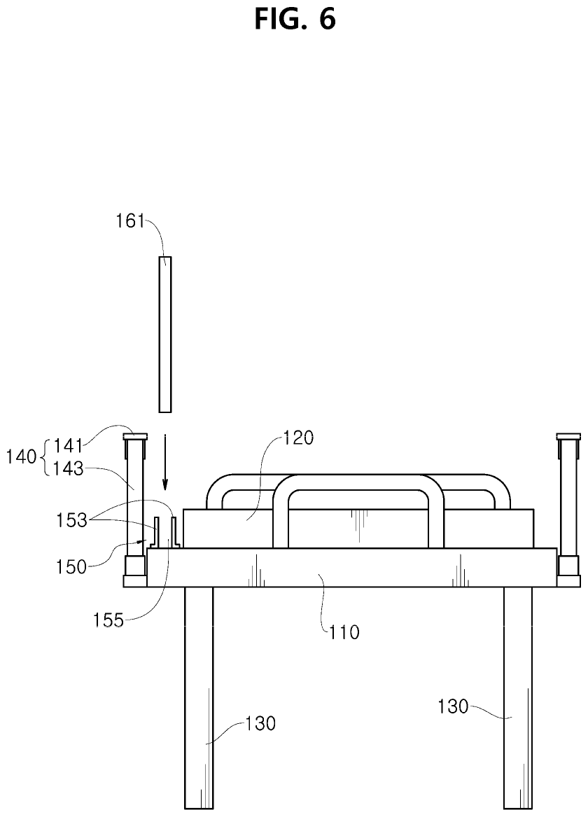

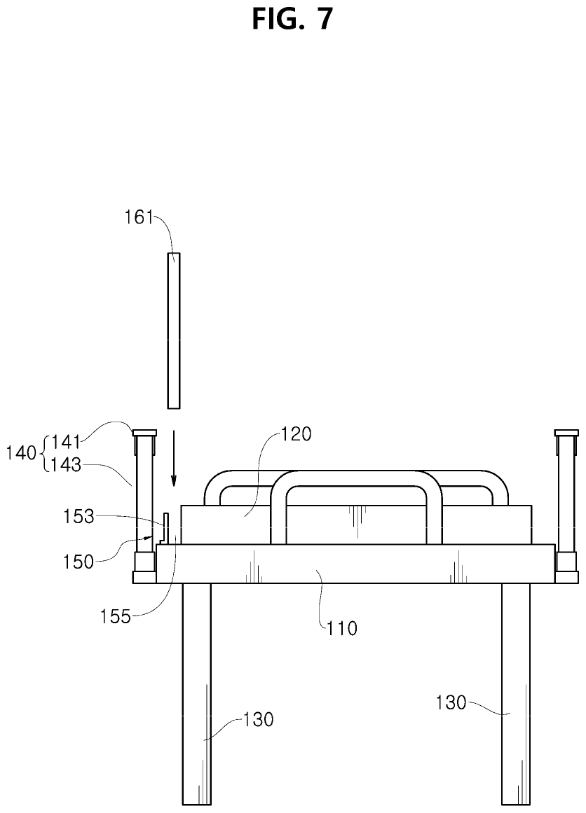

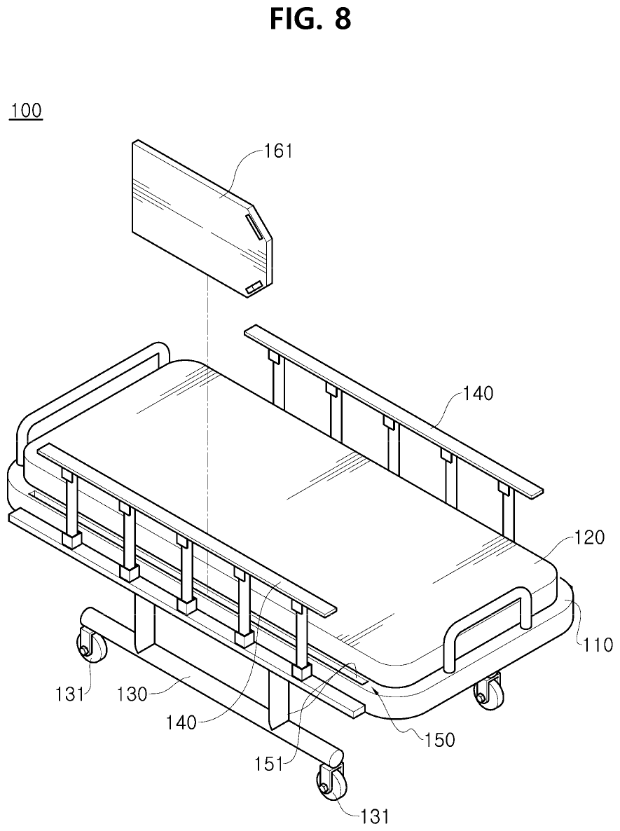

In the drawings, FIG. 2 is a perspective view showing a side-lying bed for X-ray imaging according to the present invention, FIG. 3 is a cross-sectional view of the bed in a state where a detector is mounted on a detector mounting unit shown in FIG. 2. FIG. 4 is a schematic view showing the process of taking an X-ray image while a patient is lying on his/her side on the side-lying bed for X-ray imaging shown in FIG. 2, FIG. 5 is a cross-sectional view of the bed in the state shown in FIG. 4. And FIG. 6 is a schematic view showing another example of the detector mounting unit shown in FIG. 3, FIG. 7 is a schematic view showing the other example of the detector mounting unit shown in FIG. 3, and FIG. 8 is a schematic view showing an example in which wheels are mounted so that the bed shown in FIG. 2 may be transferred.

As shown in FIG. 2, a side-lying bed for X-ray imaging 100 includes a frame 110 having a flat rectangular structure with a length and width for a patient to lie on, a mattress 120 located on the upper surface of the frame 110 on which the patient lies, a plurality of legs 130 mounted on the lower edge of the frame 110, a handrail 140 located on both sides of the frame 110 in the longitudinal direction to prevent the patient from falling and folded downward and unfolded upward, and a detector mounting unit 150 for mounting a detector 161 of an X-ray imaging apparatus in the longitudinal direction of the frame 110 on the upper side of the frame 110.

In the side-lying bed for X-ray imaging 100 configured in this way, the patient takes a side-lying posture on the mattress 120, the detector 161 is mounted on the detector mounting unit 150 located on the side facing the chest of the patient, and then the handrail 140 located on the back side of the patient is folded downward. Then, the X-ray imaging apparatus 162 is placed on the back side of the patient to perform X-ray imaging.

The side-lying bed for X-ray imaging configured as described above will be specifically described below.

As shown in FIG. 2 to FIG. 5, in the side-lying bed 100 for X-ray imaging, with reference to the frame 110 having a flat rectangular structure, a plate is fixed to an upper surface of the frame 110, the plurality of legs 130 are formed on a bottom surface of the frame 100, and the mattress 120 on which a patient may lie is positioned on the upper surface of the frame 110.

The mattress 120 is made of a material through which X-rays may pass and does not contain any materials that are difficult to pass (e.g., metallic materials) through via X-rays. The mattress 120 is typically composed of a sheath made of a synthetic resin and a sponge filled therein, and preferably has a strength such that the mattress 120 does not sag significantly even when a patient lies on the mattress 120.

The height of the frame 110 is determined according to the length of the plurality of legs 130 formed on the bottom surface of the frame 110, and if necessary, a function of adjusting the length of the legs 130, which is not shown in the drawings, may be provided to adjust the height of the frame 100.

Alternatively, as shown in FIG. 8, wheel 131 may be mounted on the lower end of the leg 130 to form a transfer bed (stretcher cart) capable of transferring the bed.

Further, detector mounting units 150 are respectively formed on both sides of the frame 110 in the longitudinal direction of the frame 110. A portable X-ray imaging apparatus is divided into a portable detector 161 and a portable X-ray imaging device 162, and the detector 161 is a device that receives and images X-rays emitted by the X-ray imaging device 162.

Therefore, when the X-ray imaging device 162 irradiates X-rays from the patient's back toward the detector 161 located on the chest side of the patient while the patient is side-lying on the mattress 120, the state of the patient is interpreted based on the image generated by the X-rays transmitted through the patient.

In this way, the detector 161 is mounted on one of the detector mounting units 150 positioned on each side of the frame 110, and the X-ray imaging device 162 is positioned on the back of a patient side-lying on a mattress 120.

The detector mounting unit 150 will be described in detail below.

The detector 161 has a rectangular plate structure having a thickness of approximately 1 to 4 cm, and when the detector 161 is attached to the detector mounting unit 150, the detector 161 stands upright in a state perpendicular to the frame 110.

To this end, the detector mounting unit 150 may be configured by a elongated groove 151 formed in the frame 110. The elongated groove 151 has a structure that is open on the upper surface and closed on both sides and the bottom, and when the lower end of the detector 161 is inserted through the open upper surface, the detector 161 fitted in the elongated groove 151 is vertically erected with respect to the frame 110.

On the other hand, as shown in FIG. 6, as another detector mounting unit 150, a pair of rails 153 may be fixed to the upper surface of the frame 110, and the detector 161 may be inserted into a gap formed between the rails 153 so that the detector 161 may be positioned upright. Here, the rails 153 are preferably an L-shaped steel, and may be configured such that flanges of the L-shaped steel face each other and a web of the L-shape steel is fixed to the upper surface of the frame 110.

As another example of the detector mounting unit 150, as shown in FIG. 7, the rail 153 may be fixed at a point spaced apart from the side surface of the mattress 120 by the thickness of the detector 161, and the detector 161 may also be configured to be positioned upright by inserting it into the gap 155 formed between the side surface of the rail 153 and the mattress 120.

In the detector mounting unit 150, the lower boundary point (B in FIG. 3) of the imaging area of the detector 161 capable of X-ray imaging must be positioned lower than the upper surface of the mattress 120. When taking an X-ray image of the chest of a patient side-lying on the upper surface of the mattress 120, the mattress 120 may be slightly pressed down due to the patient's weight. In this way, in order to take an X-ray image of the entire chest of the patient even when the mattress 120 is pressed down, the purpose is to enable X-ray imaging of the patient even when the mattress 120 is pressed down. If the lower boundary point B of the detector 161 is higher than or equal to the upper surface of the mattress 120, and the mattress 120 is pressed by the patient's weight, a part of the patient's body will be positioned below the lower boundary point B of the detector 161, thus going out of the X-ray imaging range and making it impossible to capture an image of the entire patient's chest.

On the other hand, with the patient positioned between the detector 161 mounted to the detector mounting unit 150 and the X-ray imaging device 162, the handrail 140 positioned between the patient and the X-Ray imaging device 162 should be folded down.

The handrail 140 includes an upper bar 141 positioned horizontally, and a plurality of vertical bars 143 hinged at the upper end to the upper bar 141 and hinged at the lower end to the frame 110, and includes locking means for locking the handrail 141 from collapsing in the unfolded state in the upward direction of the frame 110. In the unlocked state of the locking means the upper bar 141 moves downwards as the vertical bars 143 pivot around the hinged engagement.

At this time, in a state where the handrail 140 is folded downward, the upper bar 141 of the handrail 140 lies below the upper surface of the mattress 120. This is to prevent the X-rays irradiated by the X-ray imaging device 162 from interfering with the upper bar 141.

On the other hand, the side-lying bed 100 for X-ray imaging according to the present invention may be applied as a hospital bed that a patient normally uses, as shown in FIG. 2, by being positioned in a hospital room. In this case, the medical staff carries the portable X-ray imaging apparatus and moves to the hospital room, so that X-ray imaging may be performed in the hospital room without moving the patient.

As shown in FIG. 8, when the wheels 131 are mounted on the legs 130 of the side-lying bed for X-ray imaging 100 according to the present invention, it may be used as the transfer bed 100. In this case, after the patient is transferred from the hospital room to the transfer bed 100 and the patient is transferred to the X-ray imaging room by using the transfer bed 100, it is possible to perform X-ray imaging directly on the transfer bed 100 through the X-ray imaging device 162 fixed to the X-Ray imaging room without having to transfer the patient to the imaging table of the X-Ray imaging room. Therefore, compared to the prior art, there is no need to transfer the patient to the imaging table, which has the advantage of providing convenience to the patient and the medical staff.

Although the side-lying bed for X-ray imaging according to the present invention described above is described as X-ray imaging of only the chest of the patient in the side-lying state, this is only an example for describing the present invention in an easy-to-understand manner, and various parts such as the vertebrae of the head, the abdomen, and the legs of the patient may be photographed in a side-lying state. In addition, the side-lying bed for X-ray imaging according to the present invention may photograph various parts such as the head, spine, abdomen, and legs of a patient in various postures as well as photographing in a side-lying state.

1. A side-lying bed for x-ray imaging in which a patient may lie on his/her side, comprising:
a frame that supports a patient in a side-lying posture;
a mattress with cushions positioned on the upper surface of the frame; and
a detector mounting unit formed on one side of the frame in a longitudinal direction of the frame such that a detector for X-ray imaging may be mount on the detector mounting unit.

2. The side-lying bed for x-ray imaging of claim 1,
wherein the detector mounted on the detector mounting unit and positioned upright, and a lower boundary point of the imaging area of the detector is located below the upper surface of the mattress.

3. The side-lying bed for x-ray imaging of claim 1,
wherein a handrail formed on the other side of the frame is extended to the upper portion of the frame or folded to the lower portion of the frame, and when the handrail is folded, the handrail is located below the top surface of the mattress.

4. The side-lying bed for x-ray imaging of claim 1,
wherein the detector mounting unit is an elongated groove formed in a longitudinal direction of the frame.

5. The side-lying bed for x-ray imaging of claim 1,
wherein the detector mounting unit is a rail fixed in a longitudinal direction of the frame, and the detector is inserted and mounted in a gap between the rail and a side surface of the mattress.

6. The side-lying bed for x-ray imaging of claim 1,
wherein the detector mounting unit is fixed to the frame with two rails parallel to each other, and the detector is mounted by being inserted into the gap formed between the two rails.

7. The side-lying bed for x-ray imaging of claim 1,
wherein legs of the frame are equipped with wheels and are movable.

8. The side-lying bed for x-ray imaging of claim 1,
wherein the mattress is made of a synthetic resin capable of transmitting X-rays.

9. The side-lying bed for x-ray imaging of claim 1,
wherein the detector mounting units are formed on both sides of the frame respectively, and handrails are mounted on the outer sides of the detector mounting units on both sides respectively, and the handrails are extended to the upper portion of the frame or folded to the lower portion of the frame, and when the handrails are folded, the handrails are positioned below the upper surface of the mattress.
